(19) United States
(12) Patent Application Publication
(10) Pub. No.: US 2024/0216582 A1
(43) Pub. Date: Jul. 4, 2024

(54) ENGINEERED NERVE GRAFTS, METHODS OF MAKING THE SAME, AND METHODS OF TREATMENT USING THE SAME

(71) Applicant: Axogen Corporation, Alachua, FL (US)

(72) Inventors: Nikunj Kumar AGRAWAL, Tampa, FL (US); Curt DEISTER, Alachua, FL (US)

(73) Assignee: Axogen Corporation, Alachua, FL (US)

(21) Appl. No.: 18/531,802

(22) Filed: Dec. 7, 2023

Related U.S. Application Data
(60) Provisional application No. 63/594,656, filed on Oct. 31, 2023.
(60) Provisional application No. 63/477,486, filed on Dec. 28, 2022.

(51) Int. Cl. A61L 27/52; A61L 27/26; A61L 27/54

(52) U.S. Cl. CPC A61L 27/52; A61L 27/26; A61L 27/54; A61L 2300/236; A61L 2300/252; A61L 2430/32

(57) ABSTRACT

The present disclosure provides engineered nerve grafts, methods of making engineered nerve grafts, and methods of using an engineered nerve graft to repair a nerve. Engineered nerve grafts of the present disclosure may include a body extending from a first end to a second end, the body being formed of a biocompatible hydrogel, and a plurality of microchannels extending continuously through the body from the first end to the second end, wherein each of the plurality of microchannels may have an effective diameter of about 1 micrometer to about 200 micrometers.

### CROSS-REFERENCE TO RELATED APPLICATIONS

This patent application claims the benefit under 35 U.S.C. § 120 to U.S. Provisional Patent Application No. 63/594,656, filed on Oct. 31, 2023, and U.S. Provisional Patent Application No. 63/477,486, filed on Dec. 28, 2022, the entire contents of each of which are incorporated herein by reference.

### TECHNICAL FIELD

The present disclosure relates generally to the fields of neurobiology and medicine. More particularly, the present disclosure relates to engineered tissues, such as engineered nerve grafts, methods of making such nerve grafts, and methods of treating nerve deficits using such engineered nerve grafts.

### BACKGROUND

Nerve damage, regardless of cause, may result in significant, and in some cases severe, disability and discomfort for a subject. Neuropathic injury, in particular, can cause chronic pain, loss of sensation, loss of some or all muscle control, or other undesirable effects. Addressing the deleterious effects of peripheral nerve injury is still a considerable challenge, particularly when there is a delay in nerve repair or when axons are required to reestablish connections with peripheral targets over large nerve defects or long distances. In such cases, the regenerating axons might not have the required chemical and physiological cues to effectively regenerate and reinnervate their end-target organs. For example, relatively long nerve gaps may experience a depletion of neurotrophic factors at the proximal nerve stump, and the concentration of neurotrophic factors may decline in a growth-supportive environment in the distal nerve stump.

One potential treatment of nerve injuries is surgical intervention via autologous tissue replacement, in which nerve tissue from an uninjured region is grafted to a damaged region of a nerve. However, there are significant disadvantages associated with autologous nerve grafting, such as donor site trauma and morbidity, increased complexity of the grafting procedure, increased surgical time, scarring, and sensory loss at the donor site, among others. Further, hollow conduits have been used in lieu of grafts to provide guidance for nerve regeneration, but these conduits lack structural support for optimal nerve regeneration and may result in disorganized growth or collapse of the nerve fiber cables and thus suboptimal clinical outcomes.

Embodiments of the disclosure may overcome at least one or more of the issues described above by providing engineered nerve grafts that mimic the micro-architecture of a human nerve to promote nerve regeneration.

### SUMMARY

In accordance with the present disclosure, an engineered nerve graft may include a body extending from a first end to a second end, the body being formed of a biocompatible hydrogel, and a plurality of microchannels extending continuously through the body from the first end to the second end. Each of the plurality of microchannels may have an effective diameter of about 1 micrometer to about 200 micrometers.

In one aspect, a method of making an engineered nerve graft may include: assembling a plurality of microfibers within a liquid hydrogel so that the plurality of microfibers are generally aligned with one another; curing the liquid hydrogel to form a body of the engineered nerve graft; and removing the plurality of microfibers from the body to form a plurality of microchannels that extend from a first end of the body to a second end of the body.

In another aspect, an engineered nerve graft may be used in a method of repairing a nerve. The method of repairing a nerve may include implanting the engineered nerve graft at a nerve repair site of a subject.

Other objects, features, and advantages of the present disclosure will become apparent from the following detailed description. It should be understood, however, that the detailed description and the examples, while indicating exemplary embodiments of the present disclosure, are given by way of illustration only, since various changes and modifications within the spirit and scope of the invention will become apparent to those skilled in the art from this detailed description. Note that simply because a particular compound is ascribed to one generic formula does not mean that it cannot also belong to another generic formula.

The singular forms “a,” “an,” and “the” include plural reference unless the context dictates otherwise. The terms “substantially,” “approximately,” and “about” refer to being nearly the same as a referenced number or value. As used herein, the terms “substantially,” “approximately,” and “about” generally should be understood to encompass±10% of a specified amount or value. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

Both the foregoing general description and the following detailed description are exemplary and explanatory only and are not restrictive of the features, as claimed. As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” In addition, the term “between” used in describing ranges of values is intended to include the minimum and maximum values described herein.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of exemplary embodiments presented herein.

FIG. 1 schematically depicts an engineered nerve graft, according to one or more aspects of the present disclosure.

FIG. 2 is an image of magnified cross sections of an exemplary engineered nerve graft compared to a traditional hydrogel, according to one or more aspects of the present disclosure.

FIG. 3 is a flowchart of an exemplary method of making an engineered nerve graft, according to one or more aspects of the present disclosure.

FIG. 4 is an image of microfibers assembled within a hydrogel, according to one or more aspects of the present disclosure.

FIG. 5 is an image of the microfibers assembled within the hydrogel of FIG. 4 submerged in a solvent, according to one or more aspects of the present disclosure.

FIG. 6, FIG. 7, and FIG. 8 are images from confocal microscopy showing cells within the microchannels of segments.

FIG. 9 is an image of a control graft, without microchannels, with a DRG.

FIG. 10 is an image of an engineered nerve graft having microchannels.

FIG. 11 is an image of a control graft.

FIGS. 12-14 are images of portions of a sample of an engineered nerve graft having microchannels.

FIGS. 15-17 are images of portions of another sample of an engineered nerve graft having microchannels.

FIGS. 18-20 are images of portions of still another sample of an engineered nerve graft.

### DETAILED DESCRIPTION

Embodiments of the present disclosure are drawn to engineered nerve grafts and related methods of making and using engineered nerve grafts. As described above, current nerve grafts are harvested from a patient (in the case of autografts), from a cadaver or other source (in the case of allografts), or from another species (in the case of xenografts). Each of these graft types (collectively, harvested nerve grafts) has its own drawbacks. The engineered nerve grafts described herein provide an alternative to harvested nerve grafts and aim to mimic the structure of endoneurial tubes in harvested nerve grafts. This is achieved by introducing microchannels into a biocompatible hydrogel matrix to mimic the microstructure of a nerve, e.g., a human nerve.

Embodiments of the disclosure include a body formed of a hydrogel matrix through which microchannels extend. The microchannels may extend from a first end of the body to a second end of the body, providing scaffolding along which nerves may regenerate when implanted at a tissue repair site. Engineered nerve grafts of the disclosure may guide the re-growth of nerves along a conduit for connector assisted repair. In some aspects, engineered nerve grafts may also incorporate bioactive molecules and may function as a local drug delivery vehicle. Thus, the engineered nerve grafts of the present disclosure may promote nerve regeneration, which, in some aspects, may in turn improve subject outcomes. Exemplary engineered nerve grafts, related methods for their preparation, and related methods of treatment using the nerve grafts, are described in detail below.

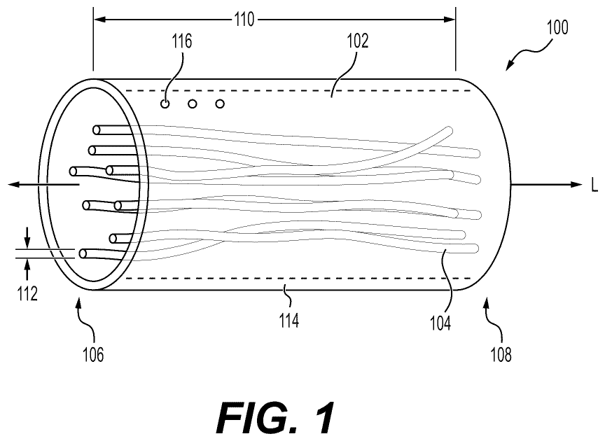

FIG. 1 schematically illustrates an engineered nerve graft 100. The engineered nerve graft 100 may include a body 102 containing microchannels 104 extending therethrough. As depicted, the body 102 may extend along a direction L from a first end 106 to a second end 108. In some embodiments, the body 102 may be substantially cylindrical, although any suitable shape may be used. The body 102 may be formed of a biocompatible material suitable for implantation in the body such that nerve axons may grow into and through the body 102 via the microchannels 104. The body 102 may have a length 110 between the first end 106 and the second end 108 that is about 1 mm to about 200 mm. For example, the body may have a length 110 that is about 1 mm to about 50 mm, about 10 mm to about 50 mm, about 15 mm to about 30 mm, about 1 mm to about 25 mm, about 5 mm to about 20 mm, about 50 mm to about 150 mm, about 50 mm to about 70 mm, about 100 mm to about 150 mm, or about 100 mm to about 120 mm. The body 102 may have a width perpendicular to direction L (e.g., a diameter) of about 0.5 mm to about 10 mm, e.g., about 0.5 mm to about 8 mm, about 1 mm to about 5 mm, e.g., about 1 mm, about 2 mm, about 3 mm, about 4 mm, or about 5 mm. The engineered nerve graft 100 may come in different lengths or different diameters to facilitate use with repairing nerve injuries of different sizes. In some aspects, the engineered nerve graft 100 may be cut to a desired length prior to use by a clinician in order to be tailored in size to the nerve repair being conducted.

As described above, the body 102 may be formed of a suitable biocompatible hydrogel material. The material from which the body 102 is formed may be biologically inert. For example, the body 102 may include a hydrogel such as polyethylene glycol diacrylate (PEGDA), hyaluronic acid (HLA), an HLA-based hydrogel, a modified HLA-based hydrogel, a collagen-based hydrogel, a gelatin-based hydrogel, a photo-crosslinked material such as lithium phenyl-2,4,6-trimethylbenzoylphosphinate (LAP) or a member of the Irgacure® family (efficient UV curing additives used to initiate the photopolymerisation of reactive prepolymers), or any other suitable biocompatible material or combination of materials. The material of which the body 102 is formed may be UV-cross-linkable.

In some aspects, the properties of the material of which the body 102 is formed or the curing process to which the hydrogel is subjected may be tailored to mimic the mechanical properties of nerve allografts or autografts, to adjust the degradation rate of the graft, to adjust the mechanical properties of the graft according to the intended use of the engineered graft, etc. For example, longer grafts may need to be present in the body for longer amounts of time to allow for nerve regeneration across the length of the graft, whereas shorter grafts may need to be present for less time. In some aspects, it may be desirable for longer or wider grafts to have more structural stability as compared to shorter or narrower grafts. Tweaking the characteristics of the grafts may be achieved, e.g., by changing the hydrogel concentration, crosslinking density, type of crosslinker, curing time (e.g., UV light curing time), UV radiation dosage, or other aspects of the composition or curing process. In some aspects, the mechanical properties of the material of which the body 102 is formed or the curing process to which the hydrogel is subjected may be tailored (e.g., as described previously) to control one or more aspects of drug delivery, e.g., the release of bioactives from the hydrogel, as will be described further below. In one example, HLA has some thermo-gelling properties, so methacrylate groups may be added to functional side chains and bonded with UV light. In some aspects, changing the concentration of methacrylate groups may allow for tailoring the properties of the body 102.

The microchannels 104 may extend from the first end 106 of the body 102 to the second end 108 of the body 102, as depicted in FIG. 1. That is, the microchannels 104 generally may extend end-to-end within the body 102. In particular, the microchannels 104 may extend continuously along the body 102 from the first end 106 to the second end 108, although it is possible that manufacturing limitations may mean that some of the microchannels 104 may not extend completely from the first end 106 to the second end 108. In embodiments, a majority of the microchannels 104, if not all of the microchannels 104, may be discrete from one another. In other words, each microchannel of the microchannels 104 may be separated from each other microchannel of the microchannels 104, although it is contemplated that manufacturing limitations may mean that some of the microchannels 104 may at least partially overlap with one another. The microchannels 104 may be generally longitudinally aligned, running end-to-end, as noted above, to mimic native nerve micro-architecture.

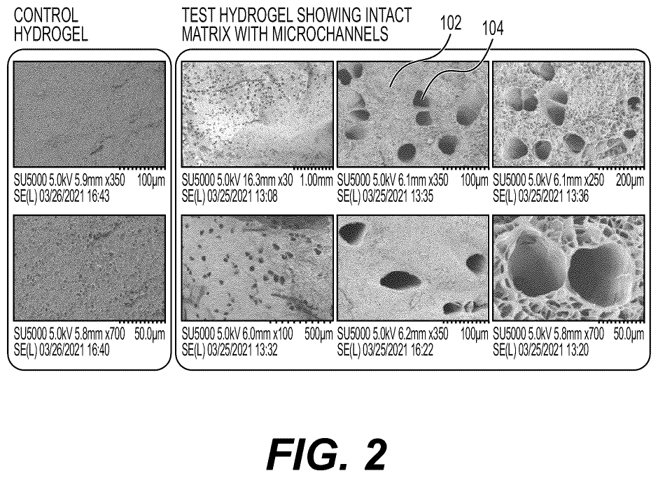

In some embodiments, the microchannels 104 may be substantially cylindrical. The microchannels 104 may be narrower than the body 102. In particular, the microchannels 104 may have an effective width, e.g., diameter 112, of about 1 micrometer to about 200 micrometers, such as about 1 micrometer to about 100 micrometers, about 10 micrometers to about 150 micrometers, about 10 micrometers to about 70 micrometers, about 40 micrometers to about 70 micrometers, about 30 micrometers to about 60 micrometers, about 40 micrometers to about 60 micrometers, about 20 micrometers to about 30 micrometers, about 10 micrometers to about 40 micrometers, or about 10 micrometers to about 30 micrometers. Accordingly, in some embodiments, the microchannels 104 may be sized similarly to the endoneurial tubes found in tissue allografts and autografts, which may have an effective width, e.g., of about 20 micrometers to about 30 micrometers. The microchannels 104 may be arranged and distributed throughout the body 102 at a density of about 1,000 to about 30,000 microchannels per square millimeter of body measured in a plane perpendicular to the lateral direction L. For example, such a density may be about 5,000 to about 20,000 microchannels per square millimeter of body, about 8,000 to about 12,000 microchannels per square millimeter of body, or about 10,000 to about 30,000 microchannels per square millimeter of body. FIG. 2, described further below, depicts magnified cross-sectional images of exemplary microchannels 104 within an engineered nerve graft 100.

In some aspects, the microchannels 104 may be aligned with one another, with each microchannel 104 extending substantially parallel to other microchannels 104. Alternatively, the microchannels 104 may extend end-to-end but may not be parallel with one another. In some embodiments, the microchannels 104 may be at a predetermined spacing from one another, while in other aspects, microchannels 104 may be randomly spaced apart from one another. If desired, alignment and spacing of the microchannels 104 may be confirmed by experimentation. The microchannels 104 may be formed within the engineered nerve graft 100 so that they constitute about 50% up to about 90% of a cross-sectional area of the engineered nerve graft 100.

Still referring to FIG. 1, the engineered nerve graft 100 may include a membrane 114. The membrane 114 may extend around at least a portion of the body 102 such that the membrane 114 fully or at least partially surrounds the body 102. The membrane 114 may extend fully or partially along the length 110 of the body 102 or may extend fully or partially along the outer perimeter (e.g., circumference) of the body 102. The membrane 114 may be made from a biocompatible material. For example, the membrane 114 may be a natural material, such as amniotic-based tissue (e.g., amniotic/chorionic membrane), reconstituted denatured collagen, dermis, fascia, pericardium, or small intestinal submucosa (SIS). In some aspects, membrane 114 may be formed of a synthetic material, such as a non-woven or woven structure, which may include homopolymers, copolymers, and/or polymeric blends of one or more of the following monomers: glycolide, lactide, caprolactone, dioxanone, trimethylene carbonate, monomers of cellulose derivatives, or monomers that polymerize to form polyesters. Additional synthetic materials that may be included instead of, or in addition to a natural material, include silicone membranes, expanded polytetrafluoroethylene (ePTFE), polyethylene tetraphthlate (Dacron), polyurethane aliphatic polyesters, poly(amino acids), poly(propylene fumarate), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and blends thereof. Natural polymers may include collagen, elastin, thrombin, fibronectin, starches, poly(amino acid), gelatin, alginate, pectin, fibrin, oxidized cellulose, chitin, chitosan, tropoclastin, hyaluronic acid, fibrin-based materials, collagen-based materials, hyaluronic acid-based materials, glycoprotein-based materials, cellulose-based materials, silks, polyglycolide (PGA), or any other biocompatible material or combination of materials. In some aspects, the membrane 114 may be a mesh. The membrane 114 may provide a structure through which the engineered nerve graft 100 may be sutured in place during nerve repair. The membrane 114 may provide mechanical support and the ability to fix the engineered nerve graft 100 in place, e.g., to suture it in place relative to proximal and distal nerve ends that the engineered nerve graft 100 is configured to be implanted near. In some embodiments, the membrane 114 may have one or more pre-made suture holes or may have pre-set sutures, e.g., sutures (not depicted), to facilitate implantation at a nerve repair site. In other aspects, the membrane 114 may allow the nerve graft 100 to more closely mimic the native environment within the body or may increase the time it takes for nerve graft 100 to degrade, e.g., by protecting from enzymes in the environment that may degrade the nerve graft 100.

The membrane 114 may be wrapped around the engineered nerve graft 100 after formation of the body 102 and/or the microchannels 104. The membrane 114 may then be secured in place, e.g., by welding the membrane 114 onto the body 102. In some aspects, the membrane 114 may be welded in place using the same or another suitable hydrogel material as is used to form the body 102. In other aspects, an adhesive may be used to maintain the membrane 114 in place, or an inner surface of the membrane 114 may be textured or may include one or more barbs, e.g., to hold the membrane 114 in place on the body 102. In other aspects, the membrane 114 may be formed as a tube, and the hydrogel of which the body 102 is formed may be poured within the membrane 114, and the body 102 may form a gel within the membrane 114, securing the membrane 114 to the body 102 during formation of the body 102.

Still referring to FIG. 1, in some embodiments, the engineered nerve graft 100 may include one or more bioactive molecules (bioactives) 116. The engineered nerve graft 100 may be functionally modified with one or more growth factors or bioactive molecules (collectively bioactives 116) to further promote nerve regeneration. In such aspects, the engineered nerve graft 100 may also function as a local drug delivery system, introducing pro-regenerative cues to promote the regeneration of axons into the network of microchannels 104. The bioactives 116 may include, e.g., laminin, collagen, collagen with HLA, PLGA, or one or more other suitable bioactives. In some aspects, human or animal peripheral nerve tissue may be solubilized and incorporated into the engineered nerve graft 100 as a bioactive 116. In other aspects, human or animal peripheral nerve tissue may be micronized into particles or into a powder for incorporation into engineered nerve graft 100 as a bioactive 116. In some aspects, homogenized nerve tissue or other bioactives 116 may be methacrolated in order to make them UV curable. The one or more bioactives 116 may encourage nerve growth through the microchannels 104.

The bioactives 116 may be distributed throughout the body 102, may be disposed on a surface of the body 102, or may be disposed within the microchannels 104, as will be discussed further below. In some embodiments, the bioactives 116 may coat or line the microchannels 104. The location of the bioactives 116 within the engineered nerve graft 100 may be selected and/or concentrated to provide for a controlled release thereof. For example, a proximal region of microchannels 104 may be loaded with bioactives configured to release at a first time to promote early growth of regenerating axons into microchannels 104, and a distal region of microchannels 104 may be loaded with bioactives configured to release at a second time, later than the first time, to promote growth of the axons as they begin to extend further into the microchannels 104. In other examples, distal regions of microchannels 104 may have a higher concentration of bioactives compared to proximal regions, so that there is still bioactive to be released by the time the axons extend further into the microchannels 104. By controlling the properties of the hydrogel, the timing, rate, and/or dose of one or more bioactives 116 may be controlled and tuned. Further, the concentration or one or more bioactives 116 in different regions of body 102 may be controlled to achieve desired release characteristics.

In embodiments in which one or more bioactives 116 are incorporated throughout the body 102, the pre-gelled or liquid hydrogel solution may be mixed with one or more bioactives 116. Upon gelling of the hydrogel solution, the one or more bioactives 116 may be incorporated throughout body 102. In other aspects, chemical cross-linking may be used to facilitate incorporation of the bioactives 116. In other aspects, surface modification may be used to incorporate the bioactives 116. For example, a sulfate-based solution with a negative charge may be used to coat at least some of the microchannels 104, and a positively charged bioactive 116 may then be coated onto the microchannels 104, e.g., by submerging the body 102 into a solution with one or more positively charged bioactives 116. In other aspects, a positively charged solution may be used to incorporate the bioactives 116. For example, a positively charged amino acid polymer, like polylysine solution, may be used to coat the microchannels 104. Following this, a negatively charged bioactive 116, such as laminin, may be coated onto the microchannels 104. As described above, the body 102 may be submerged in a solution including one or more charged base coats and/or the one or more bioactives 116 in order to coat an outer surface of the body 102 and/or the microchannels 104. In still other aspects, the bioactives 116 may be chemically or physically entrapped on or in the body 102 and/or the microchannels 104.

As will now be appreciated, the engineered nerve graft 100 may provide a nerve repair product engineered to support and encourage nerve regeneration. Embodiments of the disclosure may provide clinicians with the ability to use a hydrogel-based device for nerve repair applications and to avoid the need for an autograft, allograft, or xenograft. Accordingly, a method of using the engineered nerve graft 100 may include implanting the engineered nerve graft 100 into a nerve gap of a subject, e.g. between two damaged nerve ends of the subject. The nerve gap may have a length up to 200 mm. A length of a nerve gap may be within a range of about 1 mm to about 200 mm, within a range of about 5 mm to about 100 mm, within a range of about 10 mm to about 50 mm, within a range of about 10 mm to 30 mm, within a range of about 15 mm to about 30 mm, within a range of about 1 mm to about 25 mm, within a range of about 5 mm to about 20 mm, within a range of about 5 mm to about 30 mm, within a range of about 50 mm to about 150 mm, within a range of about 50 mm to about 70 mm, within a range about 100 mm to about 150 mm, or within a range about 100 mm to about 120 mm. The engineered nerve graft 100 may be sutured in place relative to the damaged nerve ends. This may promote the regeneration of axons into and though the microchannels 104 of the engineered nerve graft 100.

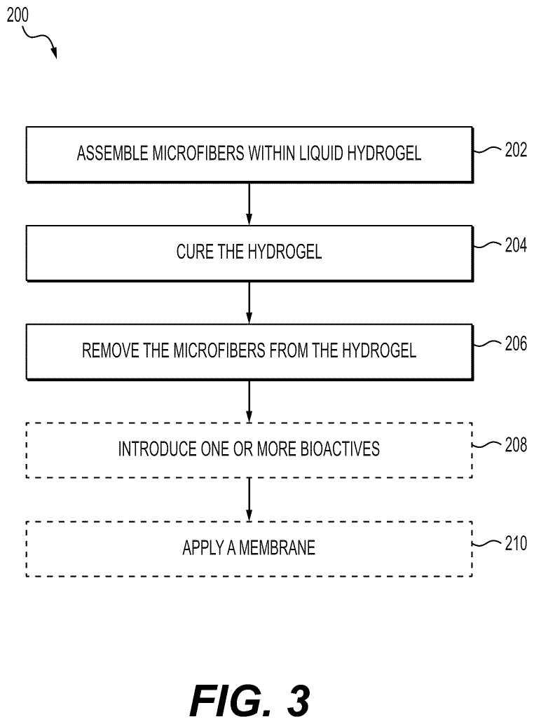

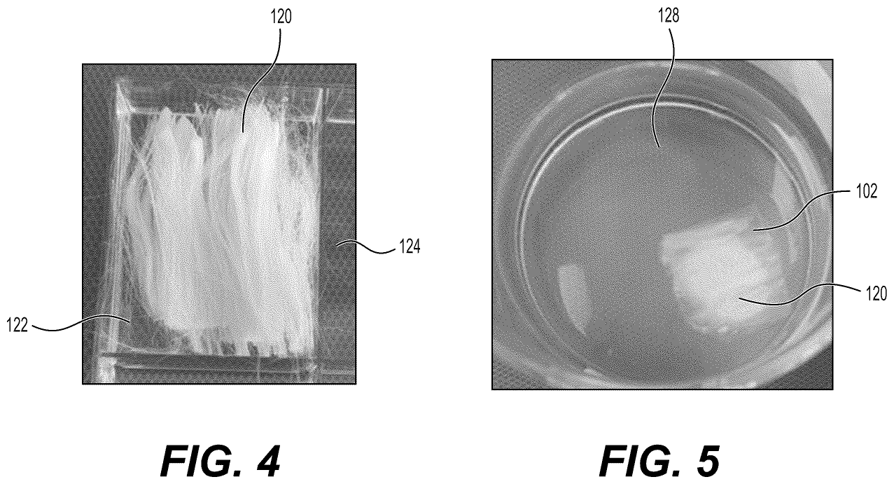

Referring now to FIGS. 3 and 4 in combination, a method 200 of making an exemplary engineered nerve graft 100 is depicted. In method 200, a hydrogel matrix may be casted around aligned microfibers 120, and then the microfibers 120 may be removed, leaving behind the hydrogel matrix with aligned microchannels 104 to form the engineered nerve graft 100.

The method 200 may include a step 202 of assembling microfibers 120 within a liquid or pre-gelled hydrogel 122. In some embodiments, the hydrogel 122 and the microfibers 120 may be assembled within a mold 124 such that the shape of the hydrogel 122 is maintained. The mold 124 may wholly or partially enclose the hydrogel 122 and the microfibers 120. In some embodiments, the mold 124 may be formed of a transparent material allowing UV light to pass through the mold 124. The mold 124 may be cylindrical in some aspects to allow the hydrogel to take a similar shape as that of a nerve. In some embodiments, the hydrogel 122 may be cast around microfibers 120.

The microfibers 120 may be generally aligned with one another so that they extend in a similar direction within the hydrogel 122. The microfibers 120 may be aligned with one another, with each microfiber 120 extending substantially parallel to other microfibers 120 or extending end-to-end adjacent one another if not parallel. In some embodiments, the microfibers 120 may be placed at a predetermined spacing from one another, while in other aspects, the spacing between microfibers 120 may be random. The microfibers 120 may have an effective width, e.g., diameter 112, that matches the effective diameters set forth above for the microchannels 104. For example, the diameter 112 of microfibers 120 may be about 1 micrometer to about 200 micrometers, about 1 micrometer to about 100 micrometers, about 10 micrometers to about 150 micrometers, about 10 micrometers to about 70 micrometers, about 40 micrometers to about 70 micrometers, about 30 micrometers to about 60 micrometers, about 40 micrometers to about 60 micrometers, about 20 micrometers to about 30 micrometers, about 10 micrometers to about 40 micrometers, or about 10 micrometers to about 30 micrometers. The microfibers 120 may be substantially aligned with one another, such as depicted in FIG. 4, within the hydrogel 122. The microfibers 120 are used as molds to form the microchannels 104 within the engineered nerve graft 100. In some aspects, the microfibers 120 may be made from a dissolvable material, such as cellulose acetate, so that the microfibers 120 may be dissolved out of the hydrogel 122, once cured. That is, the microfibers 120 may be composed of a material that can be selectively dissolved without affecting the surrounding hydrogel 122. In other embodiments, the microfibers 120 may be a plurality of sutures, e.g., 8-0 to 10-0 sutures, arranged roughly in parallel with one another. In still other embodiments, the microfibers 120 may be formed as part of a mold formed of, e.g., resin, polymer, wires, or other suitable materials. In some aspects, molds used to form the body 102 and/or the microchannels 104 may be additively manufactured, or 3D printed. As described above, the hydrogel 122 may be a suitable biocompatible hydrogel material such as one or more of PEGDA, HLA, a collagen-based hydrogel, a gelatin-based hydrogel, a fibrinogen-based hydrogel, a photo-crosslinked material such as LAP, or any other biocompatible material or combination of materials. In some embodiments, the hydrogel 122 may also include one or more bioactives 116, such as laminin, collagen, collagen with HLA, micronized nerve tissue, or one or more other suitable bioactives. The number of microfibers 120 assembled within the liquid hydrogel may approximately equal the number of microchannels 140 incorporated into the final engineered nerve graft 100 produced.

The method 200 may further include a step 204 of curing the hydrogel 122. In embodiments, this may include curing the hydrogel 122 with ultra-violet (UV) light (e.g., photo-crosslinking) or heat such that the hydrogel 122 is substantially solid. As used herein, “substantially solid” means capable of holding a shape and is not intended to imply stiffness or rigidity. The hydrogel 122 when cured may form the body 102, or a precursor of the body 102, of the engineered nerve graft 100 (depicted in FIG. 1). The step 204 of curing the hydrogel 122 with UV light may be performed for a predetermined amount of time and at a predetermined UV radiation dosage or intensity of UV light. The predetermined amount of time and the predetermined dosage for curing the hydrogel 122 may be selected based on the intensity of the UV light, the concentration of the crosslinker, or the desired mechanical properties or bioactive release profile of the resulting engineered nerve graft 100, for example, firmness or rate of degradation after implantation into the body. The predetermined amount of time may be, for example, about 5 seconds to about 10 minutes, about 10 seconds to about 10 minutes, or about 5 minutes. The UV intensity may be, for example, about 0.5 mW/cm² to about 40 mW/cm², or about 4 mW/cm². Also, prior to the step 204 of curing the hydrogel 122, the method 200 may include a step of adding one or more crosslinkers to the hydrogel 122. The amount or type of crosslinker(s) added may also be selected based on the desired mechanical properties of the resulting engineered nerve graft 100. The one or more crosslinkers may include Irgacure or Lithium phenyl-2,4,6-trimethylbenzoylphosphinate (LAP). As one specific example, LAP at a concentration of 2.5 mg/ml may be used. The LAP concentration can be varied to obtain desired mechanical properties and curing times. In a case in which one or more crosslinkers are added to the hydrogel 122, the step 204 of curing the hydrogel 122 may also be referred to as crosslinking. The predetermined amount of time may depend, at least in part, on the concentration of the one or more added crosslinkers. After the step 204 of curing the hydrogel 122, the microfibers 120 may be distributed within the solid hydrogel 122. The distribution of the microfibers 120 in the solid hydrogel 122 corresponds to the locations of the microchannels 104 within the body 102. In embodiments in which the hydrogel 122 includes one or more bioactives 116, the bioactives 116 may be dispersed through the body 102 or concentrated in one or more regions of the body 102. After the hydrogel 122 is cured, the body 102 may be removed from the mold 124.

Referring to FIGS. 3 and 5 in combination, the method 200 may include a step 206 of removing the microfibers 120 from the cured hydrogel 122 to form the microchannels 104. In some aspects, e.g., if cellulose acetate fibers are used as the microfibers 120, removing the microfibers 120 may include dissolving the microfibers 120. In other words, in step 206, the microfibers may be chemically removed from the cured hydrogel 122, leaving behind the cured hydrogel 122 including microchannels 104. If the microfibers 120 are formed of, e.g., sutures, resin, polymer, wires, or other materials, or are formed using molds or 3D printing, removal of such microfibers may be performed by mechanically pulling the microfibers out of the cured hydrogel 122, resulting in a hydrogel 122 having generally aligned microchannels 104. In some embodiments, the step 206 may optionally include trimming the ends of the body 102 to expose the microfibers 120. This may ensure that the microchannels 104 created by the microfibers 120 will extend from the first end 106 of the engineered nerve graft 100 to the second end 108 of the engineered nerve graft 100. Further, trimming the ends of the body 102 may increase exposure of the microfibers 120 to a liquid or other material used to dissolve the same. If microfibers 120 are to be dissolved out of cured hydrogel 122, then trimming to expose the microfibers 120 also allows a solution to contact the microfibers 120 in order to dissolve them.

To dissolve the microfibers 120, the step 206 may include submerging the body 102 and the microfibers 120 within a solvent 128. In particular, the step 206 may include submerging the body 102 and the microfibers 120 within the solvent 128 for at least about 2 hours, for at least about 8 hours, for at least about 12 hours, for at least about 24 hours, for at least 36 hours, or for at least 48 hours. In some aspects, the body 102 may be submerged for about 2 hours to about 48 hours, for about 2 hours to about 36 hours, or for about 8 hours to about 24 hours in order to dissolve the microfibers 120 from the body 102. The amount of time for submersion may depend, at least in part, on the type of microfibers 120 used, the type of solvent 128 used, the size of the microfibers 120 used, the quantity of the microfibers 120 used, the type of hydrogel 122 used, the size of the hydrogel 122 used, etc. The solvent 128 may be selected such that it may selectively dissolve the microfibers 120 without dissolving or negatively impacting the body 102. For example, in some embodiments, the microfibers 120 may be cellulose acetate fibers, and the solvent 128 may include acetone. In such an embodiment, the body 102 may be formed of PEGDA, for example. In such an embodiment, the acetone of the solvent 128 may dissolve or substantially dissolve the cellulose acetate of the microfibers 120 without dissolving or otherwise negatively impacting the body 102.

In some embodiments, the body 102, while submerged in the solvent 128, may be stirred or agitated to promote selective dissolution of the microfibers 120. For example, agitation of about 20 rotations per minute (RPM) to about 80 RPM, e.g., about 50 RPM, about 60 RPM, or about 70 RPM may be used.

In embodiments in which sutures are used as microfibers 120, the sutures themselves may be dissolved, if dissolvable sutures are used. In other instances, the sutures may be pulled out from the body 102. Further, if molds formed of, e.g., resin, polymer, wires, or other suitable materials, are used, then the negative molds may be removed (e.g., mechanically pulled out from body 102), leaving microchannels 104 within body 102.

As will now be appreciated, after removal of the microfibers 120, the body 102 may remain and may include the microchannels 104 described hereinabove. Accordingly, after removal of the microfibers 120, the remaining body 102 may form an engineered nerve graft 100 (as depicted in FIG. 1).

Referring to FIGS. 1 and 3 in combination, the method 200 may include an optional step 208 of coating the microchannels 104 with one or more bioactives 116. The step 208 may include first coating the microchannels 104 with a solution having a negative electrical charge, such as a sulfate-based solution. In other embodiments, the step 208 may include first coating the microchannels 104 with a solution having a positive electrical charge, such as a polylysine solution. Coating the microchannels 104 with a charged solution may involve submerging the body 102 in the charged solution, for example. The step 208 may include submerging the body 102 in one or more bioactives 116 such that the bioactives 116 coat the microchannels 104 after the submerging. If a negatively charged solution is applied before coating in one or more bioactives 116, then a positively charged bioactive 116 may then be used to coat the microchannels 104. If a positively charged solution is applied before coating in one or more bioactives 116, then a negatively charged bioactive 116, e.g., laminin, may then be used to coat the microchannels 104.

Still referring to FIGS. 1 and 3 in combination, the method 200 may include an optional step 210 of applying a membrane 114. The step 210 may include wrapping the body 102 in the membrane 114. As described above, the membrane 114 may at least partially or completely cover a circumference or a length of the body 102. After the membrane 114 is disposed on an outer surface of body 102, the membrane may be secured in place via adhesive, via curing or welding the membrane 114 in place, or by another other process. As described above, in some aspects, the membrane 114 may be welded in place using the same or another suitable hydrogel material as used for the body 102. In other aspects, an adhesive may be used to maintain the membrane 114 in place. In other aspects, an outer surface of the body 102 may be roughened, or an inner surface of the membrane 114 may be textured or may include one or more barbs, e.g., to hold the membrane 114 in place on the body 102. In other aspects, the step 210 may be performed in conjunction with the step 202 and/or the step 204. For example, the membrane 114 may be formed as a tube and may be used as a mold, and the hydrogel 122 of which the body 102 is formed may be poured within the membrane 114 during the step 202, and the body 102 may form a cured hydrogel within the membrane 114, securing the membrane 114 to the body 102. Accordingly, the membrane 114 may be fixed on the body 102 in any suitable manner. Although reference is made to one membrane 114, it is also possible that more than one layer of membrane 114 may be applied to the body 102, or that a multilayer membrane 114 is applied to the body 102.

### Example 1

Cellulose acetate fibers having a diameter of 40 to 70 micrometers were used in conjunction with a PEGDA hydrogel to form an engineered nerve graft. The cellulose acetate fibers were aligned with one another in the liquid PEGDA hydrogel, and the hydrogel was then cured using UV photo-crosslinking. After cross-linking, the ends of the cured hydrogel were trimmed to increase exposure of the cellulose acetate fibers. The cured hydrogel with cellulose acetate fibers was then submerged in acetone solution for about 24 hours at an agitation setting of 60 RPM to dissolve the cellulose acetate fibers. The six right-most images in FIG. 2 depict magnified cross-sectional views of the engineered nerve graft following dissolution of the cellulose acetate fibers. The six right-most images in FIG. 2 show the microchannels under increasing magnification from left to right. These can be compared to the images of a control hydrogel shown in the two left-most images in FIG. 2. Visual comparison shows that more defined, larger microchannels were formed in the engineered nerve grafts as compared to the control hydrogel.

### Example 2

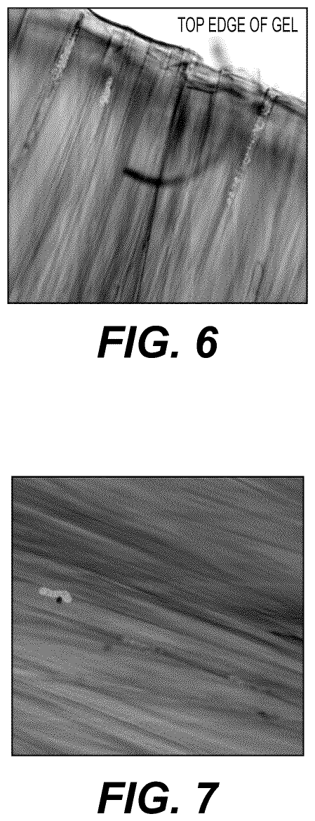

Cell migration into microchannels of an engineered nerve graft was evaluated. In particular, an engineered nerve graft was formed having a hydrogel body formed of 10% PEGDA gel with microchannels formed therein. The engineered nerve graft was cut into segments of about 3 mm in length. A diameter of fibers used to form the microchannels was between about 40 μm and 70 μm. The graft segments were transferred to a 6-well plate inside of a silicone insert, and cell media was added. The graft segments were then incubated at 37° C. for 30 minutes. The graft segments were then transferred to a 24-well plate and Neuroblastoma Stem Cells-34 (NSC-34) and additional cell media were added. NSC-34 is a hybrid cell line generated by the fusion of mouse embryonic motor neuron-enriched spinal cord cells with mouse neuroblastoma. The graft segments were then incubated at 37° C. for 24 hours, and then were imaged using phased and confocal microscopy. FIG. 6, FIG. 7, and FIG. 8 are images from confocal microscopy showing cells within the microchannels of segments. In the images of these figures, the cultured NSC-34 has a diameter of <10 μm. In particular, FIG. 6 shows a top edge of the graft segment, with green depicting phalloidin-conjugated F-actin (FITC). FIG. 7 and FIG. 8 are images with different focus planes as compared to the image of FIG. 6, and show cells present inside of multiple microchannels at different depths within a graft segment. The evaluation demonstrated that cell media of about 10 μm in diameter could migrate within microchannels of the engineered nerve graft.

### Example 3

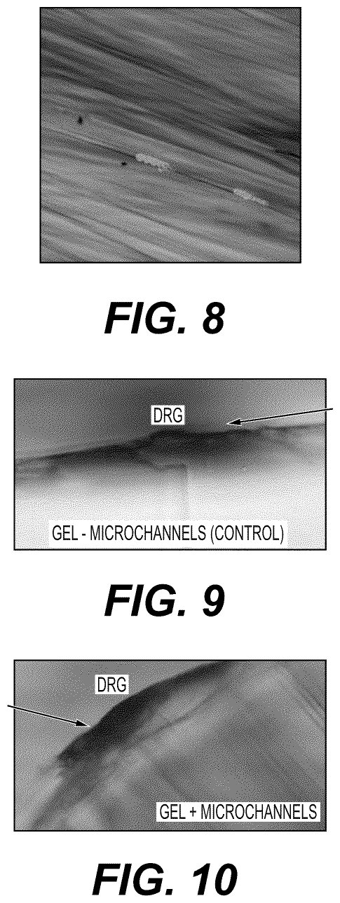

Neurite outgrowth in aligned microchannels of an engineered nerve graft formed of 10% PEGDA gel was evaluated. In particular, rat embryonic (E18) dorsal root ganglions (DRGs) were obtained and placed on engineered nerve grafts having microchannels, in accordance with the invention, with bodies of the grafts being formed of 10% PEGDA gel. The DRGs and grafts were incubated at 37° C., in 5% CO₂, for 7 days. Following incubation, the grafts were fixed in 10% formalin, stained with BIII tubulin antibody, and imaged using phase and confocal microscopy. FIG. 9 is an image of a control graft, without microchannels, with a DRG placed thereon, and FIG. 10 is an image of an engineered nerve graft having microchannels, as described above. As shown in FIG. 9, no neurite extension occurred through the hydrogel matrix of the control graft, and as shown in FIG. 10, neurite extension occurred through the microchannels in the engineered nerve graft. FIGS. 9 and 10 thus indicate that the microchannels allowed for neurite extension within them and promoted growth of neurites into the engineered nerve graft.

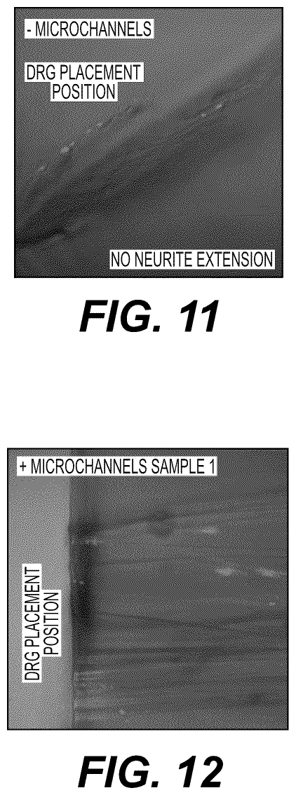

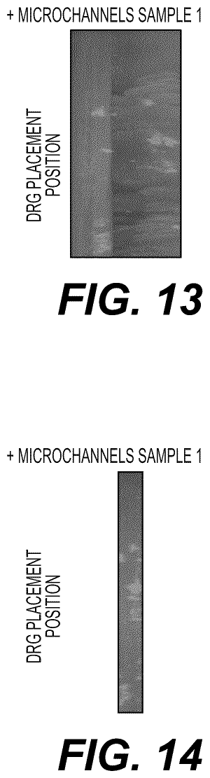

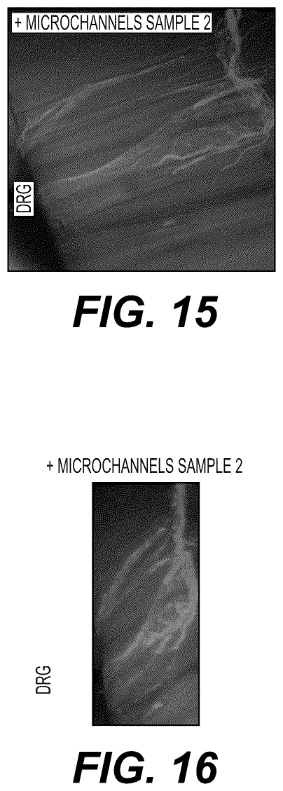

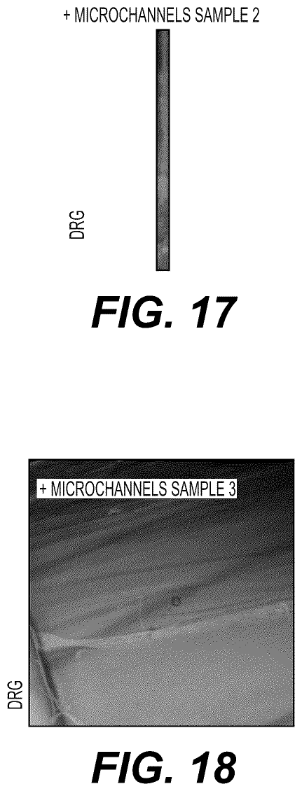

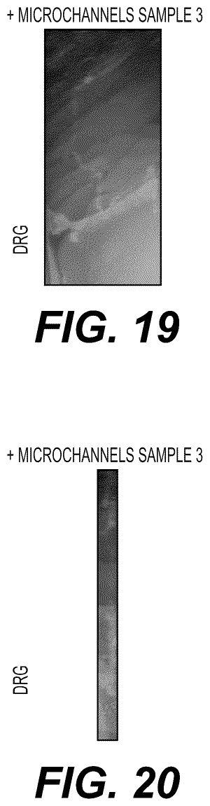

FIGS. 11-20 are images, based on screenshots of a video, of a control graft without microchannels and of three sample engineered nerve grafts having microchannels, respectively. FIG. 11 is an image of the control graft, showing no neurite extension within the hydrogel matrix in the absence of microchannels. FIGS. 12-14 are images of portions of one sample (sample 1) of an engineered nerve graft having microchannels, depicting, in red stain, neurite extension through the microchannels thereof. FIGS. 15-17 are images of portions of another sample (sample 2) of an engineered nerve graft having microchannels, depicting, in red stain, neurite extension through the microchannels thereof. FIGS. 18-20 are images of portions of still another sample (sample 3) of an engineered nerve graft, depicting, in red stain, neurite extension through the microchannels thereof. The evaluation demonstrated that neurite extension from a DRG occurred within microchannels of the engineered nerve graft, whereas no neurite extension occurred within the hydrogel of the control graft lacking microchannels. Thus it could be inferred that the neurite extension occurred within the microchannels as opposed to into the hydrogel, and thus that the hydrogel is capable of separating the individual microchannels from one another to deter or prevent cross-talk between the microchannels. The evaluation also demonstrated that neurite extension did not occur on an outer surface of the engineered nerve graft.

By virtue of the engineered nerve graft 100 and the related method 200 described herein, a biocompatible matrix with the micro-architecture akin to a human nerve is provided to allow for nerve regeneration. More specifically, uniaxially aligned 3-dimensional microchannels, formed by mechanical extraction or chemical and/or enzymatic dissolution of microfibers in a pro-regenerative hydrogel matrix, are used to form the biocompatible matrix that mimics the micro-architecture of a human nerve. Such a nerve graft may be used, for example, in the field of nerve repair, specifically to repair or fill a short to mid-size gap within a nerve. In addition, engineered nerve grafts according to the present disclosure may be relatively longer and/or thicker (that is, may have relatively greater diameters) as compared to autografts and allografts. Further, engineered nerve grafts formed of biocompatible hydrogels, as described herein, can be used as an alternative to autografts and allografts to aid in the regeneration of damaged tissue, including, for example, nerve tissue. Still further, engineered nerve grafts, as described herein, may provide for improved clinical outcomes as compared to grafts which employ hollow conduits, because the aligned microchannels mimic the micro-architecture of a human nerve and provide supportive structure for nerve regeneration through the entire length of the respective graft. In other words, the engineered nerve grafts according to this disclosure provide end-to-end support for nerve regeneration through the grafts.

In some aspects, engineered nerve grafts may be designed to offer characteristics that are not available in naturally occurring autografts, allografts, or xenografts. For example, grafts according to the disclosure may be engineered in wider diameters or longer lengths than would be found naturally. Engineered grafts may come in shapes that may not be found in nature, e.g., branched configurations or curved profiles. They may be impregnated with one or more bioactives or may have controlled degradation profiles. The mechanical properties of the engineered grafts may be tailed so that microchannels-which are meant to mimic endoneurial tubes in nerve grafts—may be distributed across a wider cross-section of the engineered graft than would be found in natural nerves. Accordingly, whereas only a portion of a natural nerve graft would be available to provide scaffolding for nerve regeneration, a larger portion of the engineered nerve graft, or almost all of the engineered nerve graft, may be available to provide scaffolding for nerve regeneration.

Also, by virtue of the engineered nerve graft 100 according to the present disclosure, and the use of a biocompatible material to form the membrane 114, for example, interference with nerves (or neurites) that grow within and around the engineered nerve graft following implantation into a subject may be deterred, if and when the membrane degrades over time.

It should be understood that although the present disclosure has been made with reference to preferred embodiments, exemplary embodiments, and optional features, modifications and variations of the concepts herein disclosed may be resorted to by those skilled in the art, and that such modifications and variations are considered to be within the scope of this disclosure as defined by the appended claims. The specific embodiments and examples provided herein are examples of useful embodiments of the present disclosure and are non-limiting and illustrative only. It will be apparent to one skilled in the art that the present disclosure may be carried out using a large number of variations of the devices, device components, methods, and steps set forth in the present description. As will be recognized by one of skill in the art, methods and devices useful for the present methods can include a large number of various optional compositions and processing elements and steps.

What is claimed is:

1. An engineered nerve graft comprising:
a body extending from a first end to a second end, the body being formed of a biocompatible hydrogel; and
a plurality of microchannels extending continuously through the body from the first end to the second end, wherein each of the plurality of microchannels has an effective diameter of about 1 micrometer to about 200 micrometers.

2. The engineered nerve graft of claim 1, wherein the plurality of microchannels each have an effective diameter of about 1 micrometer to about 100 micrometers.

3. The engineered nerve graft of claim 1, wherein the plurality of microchannels are arranged at a density within the body of about 1,000 to about 30,000 microchannels per square millimeter of the body.

4. The engineered nerve graft of claim 1, further comprising a membrane at least partially extending around an outer surface of the body.

5. The engineered nerve graft of claim 1, further comprising one or more bioactive molecules distributed through the body.

6. The engineered nerve graft of claim 1, further comprising one or more bioactive molecules disposed within at least some of the plurality of microchannels.

7. The engineered nerve graft of claim 1, further comprising one or more bioactive molecules, wherein the one or more bioactive molecules comprise one or more of laminin, collagen, or collagen with hyaluronic acid.

8. The engineered nerve graft of claim 1, wherein the body comprises one or more of polyethylene glycol diacrylate, hyaluronic acid, a collagen-based hydrogel, or a gelatin-based hydrogel.

9. The engineered nerve graft of claim 1, wherein the body comprises a photo-crosslinked hydrogel.

10. A method of making an engineered nerve graft, the method comprising:
assembling a plurality of microfibers within a liquid hydrogel so that the plurality of microfibers are generally aligned with one another;
curing the liquid hydrogel to form a body of the engineered nerve graft; and
removing the plurality of microfibers from the body to form a plurality of microchannels that extend from a first end of the body to a second end of the body.

11. The method of claim 10, wherein the plurality of microfibers each have an effective diameter about 1 micrometer to about 200 micrometers.

12. The method of claim 10, wherein the plurality of microchannels are formed within the body at a density of about 1,000 to about 30,000 microchannels per square millimeter of the body.

13. The method of claim 10, wherein the plurality of microfibers are formed of one or more of: dissolvable fibers, sutures, resin, polymer, or wires.

14. The method of claim 10, wherein the plurality of microfibers are cellulose acetate fibers.

15. The method of claim 10, wherein removing the plurality of microfibers comprises dissolving the plurality of microfibers.

16. The method of claim 15, wherein dissolving the plurality of microfibers comprises submerging the body in a solvent comprising acetone.

17. The method of claim 10, wherein removing the plurality of microfibers comprises mechanically pulling the plurality of microfibers out of the body.

18. The method of claim 10, wherein curing the hydrogel comprises photo-crosslinking the hydrogel.

19. The method of claim 10, further comprising coating at least some of the plurality of microchannels with one or more bioactive molecules.

20. The method of claim 19, wherein coating at least some of the plurality of microchannels comprises submerging the body in a solution comprising the one or more bioactive molecules.

21. The method of claim 10, further comprising applying a membrane to an outer surface of the body.

22. A method of repairing a nerve using an engineered nerve graft, the method comprising:
implanting the engineered nerve graft at a nerve repair site of a subject, the engineered nerve graft comprising:
a body extending from a first end to a second end, the body being formed of a biocompatible hydrogel; and
a plurality of microchannels extending through the body from the first end to the second end, wherein each of the plurality of microchannels has an effective diameter of about 1 micrometer to about 200 micrometers.

23. The method of claim 22, wherein the plurality of microchannels are arranged at a density of about 1,000 to about 30,000 microchannels per square millimeter of the body.

24. The method of claim 22, further comprising suturing the engineered nerve graft in place at the nerve repair site.

25. The method of claim 24, wherein the engineered nerve graft further comprises a membrane at least partially extending around an outer surface of the body, and wherein suturing the engineered nerve graft in place comprises suturing through the membrane.
